(19) United States
(12) Patent Application Publication
(10) Pub. No.: US 2013/0102975 A1
(43) Pub. Date: Apr. 25, 2013

(54) BLOOD DELIVERY SYSTEM

(71) Applicant: Julie Elisabeth Lamb, Potomac, MD (US)

(72) Inventor: Julie Elisabeth Lamb, Potomac, MD (US)

(21) Appl. No.: 13/655,579

(22) Filed: Oct. 19, 2012

Related U.S. Application Data
(60) Provisional application No. 61/549,389, filed on Oct. 20, 2011.

(51) Int. Cl. A61M 39/10; A61M 39/28

(52) U.S. Cl. CPC A61M 39/105; A61M 39/28
USPC 604/250; 604/533

(57) ABSTRACT

A device for providing blood and/or fluid to a patient is provided. The device includes a dispensing end, a blood pathway and a fluid pathway. An end cap is at the fluid dispensing end. A joining member is located proximate to the end cap and joining the blood pathway and the fluid pathway. A first group of medical components is disposed along the blood pathway, including at least one clamp, a blood filter, and a pump adaptor. A a second group of medical components is disposed in the fluid pathway, including at least one clamp, and a pump adaptor. The joining member is located downstream from the first and second groups of medical components and upstream from the end cap.

### CROSS REFERENCE TO RELATED APPLICATIONS

The instant application claims priority to provisional patent application 61/549,389, filed Oct. 20, 2011, entitled BLOOD DELIVERY SYSTEM, the contents of which are expressly incorporated by reference herein in its entirety.

### BACKGROUND OF THE INVENTION

1. Field of the Invention

The present invention relates to a blood delivery product for providing blood to patients. More specifically, the present invention relates to a dual tubing architecture and methodology for administering blood to patients.

2. Background Information

The need to provide blood to patients is well known. Various devices are designed to transfer blood and/or a fluid from bags into a patient. The fluid is typically normal saline (hereinafter “saline”). The saline can be used to prime the delivery system before providing blood, to combine with the blood, and/or as an independent fluid administered to the patient.

Known devices and methods for providing blood and saline have various drawbacks. For example, if the patient has an adverse reaction to the blood, the blood flow must be discontinued and saline must be administered to the patient. This process results in the loss of the blood/saline delivery device. There can also be a delay in providing fresh saline, as common practice is to utilize a fresh bag of saline and fresh tubing, which must be retrieved from storage and primed before it can be administered to a patient.

### SUMMARY

### Brief Description of the Drawings

FIG. 1 is a blood delivery system according to the prior art.

FIG. 2 is a blood delivery system according to an embodiment of the invention.

FIG. 3 is a blood delivery system according to another embodiment of the invention.

### DETAILED DESCRIPTION

It is to be understood that the figures and descriptions of embodiments of the present disclosure have been simplified to illustrate elements/steps relevant for a clear understanding of the present disclosure, while eliminating, for the purpose of clarity, other elements/steps found or used in typical presentations, productions, data delivery, computing systems, devices and processes. Those of ordinary skill in the art may recognize that other elements and/or steps are desirable and/or required in implementing embodiments of the present disclosure. However, because such elements and steps are well known in the art, and do not facilitate a better understanding of the present disclosure, a discussion of such elements/steps is not provided herein.

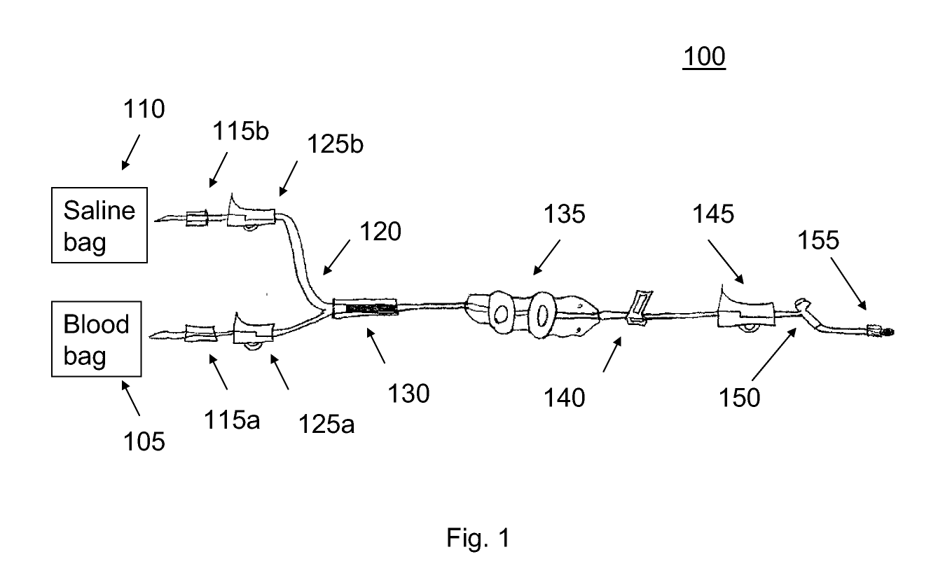

Referring now to FIG. 1, a blood delivery system 100 is shown for providing blood and/or saline from a blood bag 105 and a saline bag 110. A first spike 115a with a protective cap is configured to connect with blood bag 105, and a second spike 115b with a protective cap is configured to connect with saline bag 110. Fluid tubes from the two spikes 115a and 115b converge at a connection 120. Connection 120 converges the blood and saline pathways into a single common pathway. Clamps 125a and 125b, preferably roller clamps, control the flow of fluid from the bags 110/105 to the connection 120.

Downstream from the convergence point is a blood filter 130 (the convergence may be part of the blood filter 130), a pump adaptor 135, a pump clamp 140, a clamp 145 (preferably a roller clamp), a medication port 150, and finally an end cap 155 (which may be a so-called needless connector). As is known in the art, a connector (not shown) connects between the cap 155 and the catheter (not shown) that is connected to the patient.

The nature and function of the above components are known to those of skill in the art of blood delivery and transfusions. An accepted methodology for their use is provided in “Blood and Blood product Administration,” Overlook Hospital Nursing Administration, Issued Jul. 7, 1988, as updated Dec. 10, 2008, the contents of which are incorporated herein in its entirety.

By way of summary, saline from saline bag 110 flows through the length of the saline pathway and the common pathway, a process known as “priming.” Once primed, clamp 125b is closed to end the flow of saline while clamp 125a is opened to permit the flow of blood. Pressure is applied to blood filter 130, which draws blood from blood bag 105 into the filter to initiate the blood flow. Pump adaptor 134 and pump clamp 140 are then inserted into a pump (not shown), which regulates the extraction of blood from blood bag 105 into the patient. Clamp 145 provides a method for stopping all fluid flow from the bags 105/110 into the patient, and medication port 150 provides an independent pathway for injecting another fluid into the common pathway.

A drawback of the above device 100 is that if the patient has an adverse reaction to the blood (such as through a blood type mismatch), then the blood flow must be discontinued and replaced with a flow of saline. The entire blood delivery system 100 must be removed and disposed of, with probable corresponding loss of the contents of blood bag 110 and saline bag 105. A new saline bag 105 with tubing must be obtained from storage and primed before the saline can be provided to the patient.

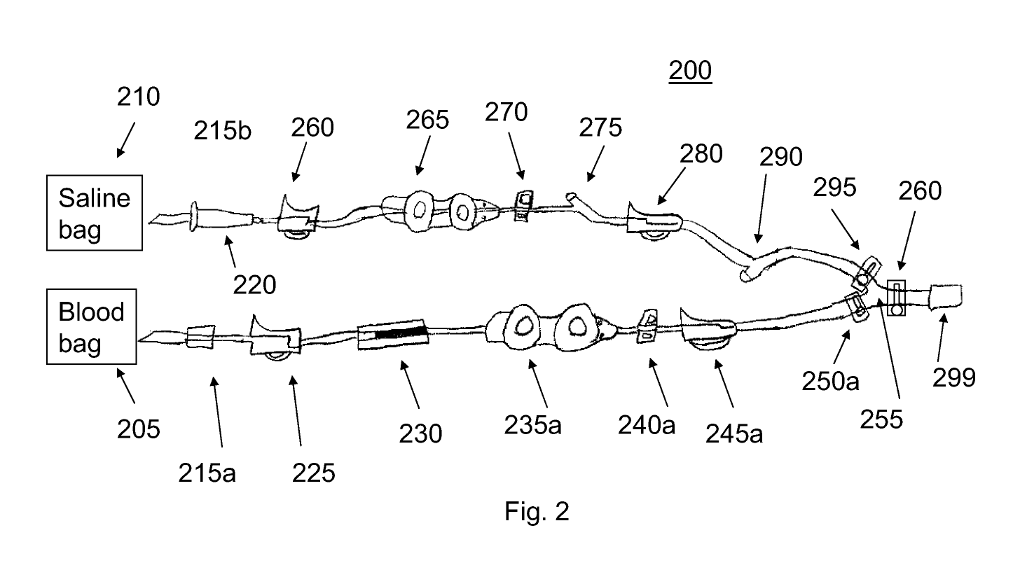

Referring now to FIG. 2, an embodiment of blood delivery system 200 is shown for providing blood and/or saline from a blood bag 205 and a saline bag 210. As can be seen compared to FIG. 1, the pathways for the blood and saline remain separate for the bulk of the device until converging proximate to the end of the device 200.

The blood pathway in FIG. 2 is similar to that in FIG. 1, and comprises a series of tubes interspaced and/or connected to various medical components. A first spike 215a with protective cap is configured to connect with blood bag 205, Clamp 225a, preferably a roller clamp, controls the flow of blood from the bags 205. Downstream from the clamp 235 is a blood filter 230, a pump adaptor 235a, a pump clamp 240a, a clamp 245a (preferably a roller clamp), a clamp 250 (preferably a slide clamp), a Y connection 255 to merge the blood and saline pathways into a common pathway, and finally a common clamp 260 configured to control the overall flow of fluid into the patient through end cap 299.

The saline pathway comprises a series of tubes interspaced and/or connected to various medical components. The pathway begins with a second spike 215b with protective cap that is configured to connect with saline bag 210. Downstream from spike 215b is drip chamber 220, a clamp 260 (preferably a roller clamp), a pump adaptor 265, a pump clamp 270, a first medication port 275, a clamp 280 (preferably a roller clamp), a second medication portion 290, and a slide clamp 295. Tubing extending from slide clamp 295 connects with Y connection 255 to merge the blood and saline pathways.

The apparatus 200 is initially primed with saline from saline bag 210. This is achieved by first opening the various clamps in the saline pathway and common pathway while keeping the clamps in the blood pathway closed. Once the saline pathway and common pathway are filled with saline, the common clamp 260 is closed, and any clamps in the blood pathway downstream from clamp 225 are opened. Saline then flows to fill the available blood pathway. Clamp 295 is then closed to seal off the saline flow, clamp 299 is opened to allow fluid flow to the patient, and clamp 205 is opened to facilitate blood flow. A pump works in cooperation with pump adaptor 235a and pump clamp 240 will be placed inside a pump (not shown) to control the rate of blood flow.

If the patient experiences a reaction to the blood, the blood flow can be stopped by closing clamp 250. Saline can then be immediately provided by opening clamp 295. Unlike the embodiment of FIG. 1, the existing tubing can be used to provide saline from saline bag 210. There is no need to replace the delivery system as in FIG. 1. Nor is there any need to locate and/or prime a new saline bag.

The size and constructions of the initial components are known to those of skill in the art of blood delivery systems. Clamp 260 is preferably far enough from end cap 299 to visually confirm whether or not any fluid is present and/or flowing in the adjacent portion of the fluid tube. Otherwise, the location and orientation of the components are known in the art. Also as known in the art, not all components are necessary.

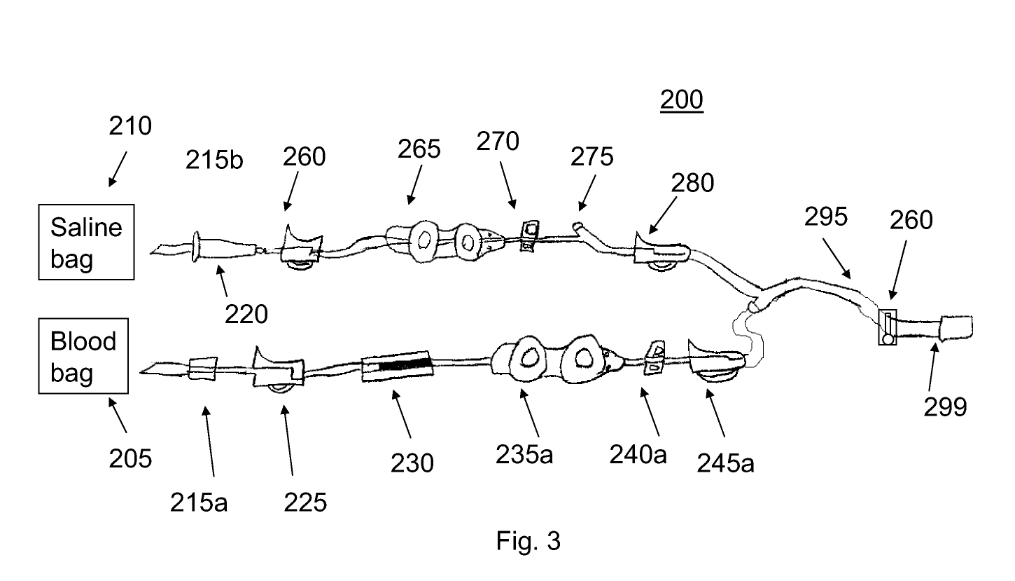

Referring now to FIG. 3, another embodiment of the invention is shown. Instead of an independent Y connection, the blood pathway can connect into the saline pathway through a medication port, e.g., medication port 290 via a known connector (not shown). Other/additional medication ports at other locations could also be used. In this embodiment, once a blood reaction is detected, the flow of blood can be physically stopped by removing the connection between the blood pathway and the medication port.

It will be apparent to those skilled in the art that modifications and variations may be made in the systems and methods of the present disclosure without departing from the spirit or scope of the invention. It is intended that the present disclosure cover the modifications and variations of this invention provided they come within the scope of the appended claims and their equivalents.

What is claimed is:

1. A device for providing blood and/or fluid to a patient, the device having a fluid dispensing end, comprising:
a blood pathway and a fluid pathway;
an end cap at the fluid dispensing end;
a joining member located proximate to the end cap and joining the blood pathway and the fluid pathway;
a first group of medical components disposed along the blood pathway, including at least one clamp, a blood filter, and a pump adaptor;
a second group of medical components disposed in the fluid pathway, including at least one clamp, and a pump adaptor; and
the joining member being located downstream from the first and second groups of medical components and upstream from the end cap.

2. The device of claim 1, further comprising a saline source connected to the fluid pathway upstream of the second group of medical components.

3. The device of claim 1, further comprising a blood source connected to the blood pathway upstream of the first group of medical components.

4. The device of claim 1, when the end cap comprises a needle-less connector.

5. The device of claim 1, wherein the joining member is three-prong Y pathway, and the device further comprises a clamp on each prong.

6. The device of claim 1, further comprising a first medication port in the fluid pathway upstream from the joining member and downstream from the second group of medical components.

7. The device of claim 6, further comprising a second medication port in the fluid pathway upstream from the first medication portion and downstream from the second group of medical components, the device further comprising a clamp in the fluid pathway between the first and second medication ports.

8. The device of claim 1, wherein the joining member is defined by a medication port in the fluid pathway, and the blood pathway is connected to the medication port.

9. A device for providing blood and fluid to a patient, the device having a dispensing end, comprising:
a blood pathway and a fluid pathway;
an end cap at the dispensing end;
a joining member located proximate to the end cap and joining the blood pathway and the fluid pathway;
a first group of medical components disposed along the fluid pathway, including in order a spike, a clamp, a pump adaptor, a clamp, a first medication port, and a clamp;
a second group of medical components disposed along the blood pathway, including in order a spike, a clamp, a blood filter, a pump adaptor, and at least one clamp; and
the joining member being located downstream from the first and second groups of medical components and upstream from the end cap.

10. The device of claim 9, further comprising a saline source connected to the fluid pathway upstream of the first group of medical components.

11. The device of claim 9, further comprising a blood source connected to the blood pathway upstream of the second group of medical components.

12. The device of claim 9, when the end cap comprises a needle-less connector.

13. The device of claim 9, wherein the joining member is three prong Y pathway, and the device further comprises a clamp on each prong.

14. The device of claim 9, further comprising a first medication port in the fluid pathway upstream from the joining member and downstream from the second group of medical components.

15. The device of claim 14, further comprising a second medication port in the fluid pathway upstream from the first medication port and downstream from the second group of medical components, the device further comprising a clamp in the fluid pathway between the first and second medication ports.

16. The device of claim 9, wherein the joining member is defined by a medication port in the fluid pathway, and the blood pathway is connected to the medication port.
